(19) United States
(12) Patent Application Publication
(10) Pub. No.: US 2021/0401501 A1
(43) Pub. Date: Dec. 30, 2021

(54) SYSTEM AND METHOD FOR RECOMMENDING PARAMETERS FOR A SURGICAL PROCEDURE

(71) Applicant: Surgical Theater, Inc., Mayfield Village, OH (US)

(72) Inventors: MORDECHAI AVISAR, HIGHLAND HEIGHTS, OH (US); Alon Yakob Geri, Orange Village, OH (US); Gidi Navrotzky, Binyamina (IL)

(21) Appl. No.: 16/930,213

(22) Filed: Jul. 15, 2020

Related U.S. Application Data
(60) Provisional application No. 62/874,307, filed on Jul. 15, 2019.

(51) Int. Cl. A61B 34/10; A61B 34/00; A61B 90/00; G02B 27/01; G06N 20/00; G16H 20/40; G16H 50/70

(52) U.S. Cl. CPC A61B 34/10; A61B 34/25; A61B 90/36; G02B 27/017; A61B 2090/365; G16H 20/40; G16H 50/70; A61B 2034/256; A61B 2034/107; G06N 20/00

(57) ABSTRACT

An artificial intelligence surgical planning system is configured to receive as input historical surgical procedure data relating to a plurality of surgical procedures previously performed for a plurality of patients; generate a surgical procedures parameters algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data; receive current surgical procedure data for a patient for which a surgical procedure is to be performed; apply the generated surgical procedures parameters algorithm to the received current surgical procedure data in order to identify a recommended surgical parameter for the surgical procedure to be performed for the current patient; and output the recommended surgical parameter to the display.

### CROSS-REFERENCE TO RELATED APPLICATION

This application claims priority from U.S. provisional patent application Ser. No. 62/874,307 filed on Jul. 15, 2019 which is incorporated by reference herein in its entirety.

### FIELD OF DISCLOSURE

The present disclosure relates to the field of surgical procedures and more specifically to the field of artificial intelligence assisted surgery.

### BACKGROUND

Surgical procedures are commonly performed by trained medical professionals to address a variety of patient needs. For example, brain surgery may be performed to remove a tumor, heart bypass surgery may be performed to improve blood flow in the coronary artery, or spinal surgery may be performed to relieve back pain. In order to perform these surgical procedures, various parameters must first be determined. For example, where to make an incision and how large of an incision to make must often be determined prior to beginning a procedure. Properly selecting these parameters may result in a successful outcome and a faster recovery time, for example. However, incorrectly selecting parameters may result in slower recovery time or complications requiring additional hospital visits and surgical procedures.

Performing brain surgery, in particular, requires first performing a craniotomy in which part of the bone of the skull is removed to expose the brain. Before performing the craniotomy, the surgeon must select a proper approach including a trajectory for reaching the brain tumor inside the brain. Based on this trajectory, the surgeon must also select an entry point in the skull as well as the size and shape of the entry point which should be exposed.

To determine such parameters for surgical procedures, the surgeon commonly begins by reviewing medical images such as x-rays, MRIs, and CT-scans. The surgeon then determines the parameters based on the review of the medical images and based on his/her individual training and experience. However, if a surgeon has limited experience or inadequate training, his selection of the parameters may not result in an optimal outcome. Moreover, because the analysis of a medical image may be at least in part a subjective process, multiple surgeons with similar training and experiences may still select parameters which slightly differ, some of which may not result in an optimal outcome.

### SUMMARY

An artificial intelligence surgical planning system includes a display and a computer having one or more processors, one or more computer-readable tangible storage devices, and program instructions stored on at least one of the one or more storage devices for execution by at least one of the one or more processors. The program instructions are configured to receive as input historical surgical procedure data relating to a plurality of surgical procedures previously performed for a plurality of patients; generate a surgical procedures parameters algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data; receive current surgical procedure data for a patient for which a surgical procedure is to be performed; apply the generated surgical procedures parameters algorithm to the received current surgical procedure data in order to identify a recommended surgical parameter for the surgical procedure to be performed for the current patient; and output the recommended surgical parameter to the display.

A method for identifying a recommended surgical parameter for a surgical procedure includes the steps of: receiving as input historical surgical procedure data relating to a plurality of surgical procedures previously performed for a plurality of patients; generating a surgical procedures parameters algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data; receiving current surgical procedure data for a patient for which a surgical procedure is to be performed; applying the generated surgical procedures parameters algorithm to the received current surgical procedure data in order to identify a recommended surgical parameter for the surgical procedure to be performed for the current patient; and outputting the recommended surgical parameter to a display.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example AI Surgical Planning system.

FIG. 2 illustrates an example AI Surgical Planning system.

FIG. 3 illustrates an example AI Surgical Planning system

FIG. 4 illustrates an example AI Surgical Planning system.

FIG. 5 illustrates an example method for recommending parameters for a surgical procedure.

FIG. 6 illustrates an example computer implementing the example AI Surgical Planning system of FIGS. 1-4.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. Pat. No. 8,311,791, incorporated in this application by reference, has been developed to convert static CT and Mill medical images into dynamic and interactive multi-dimensional full spherical virtual reality, six (6) degrees of freedom models (“MD6DM”) based on a prebuilt SNAP model that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is rendered in real time using a SNAP model built from the patient's own data set of medical images including CT, Mill, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, Mill, etc. takes a real organism and deconstructs it into hundreds of thin slices built from thousands of points, the MD6DM reverts it to a 3D model by representing a 360° view of each of those points from both the inside and outside.

Described herein is an AI Surgical Planning system, leveraging a prebuilt MD6DM model, that implements machine learning and artificial intelligence algorithms to identify recommendations for parameters in preparation for performing a surgical procedure and to communicate the identified recommended parameters via the MD6DM model. In particular, the AI Surgical Planning system includes two subsystems: a first sub-system that learns from historical data; and a second subsystem to identify and recommend one or more parameters or approaches based on the learning. Parameters may include, for example, a suggestion or recommendation as to where and how to make an incision and how large of an incision to make for a surgical procedure. It should be appreciated that although the AI Surgical Planning system is described as two distinct subsystems, the AI Surgical Planning system can also be implemented as a single system incorporating the functions and features described with respect to both subsystems. It should be further appreciated that although the examples described herein may refer specifically to performing a craniotomy and to identifying specific parameters such as identifying an entry point and a trajectory for performing a craniotomy, the example AI Surgical Planning system may similarly be used to determine an entry point and a trajectory for other surgical procedures or to determine any other type of parameter for any type of surgical procedure.

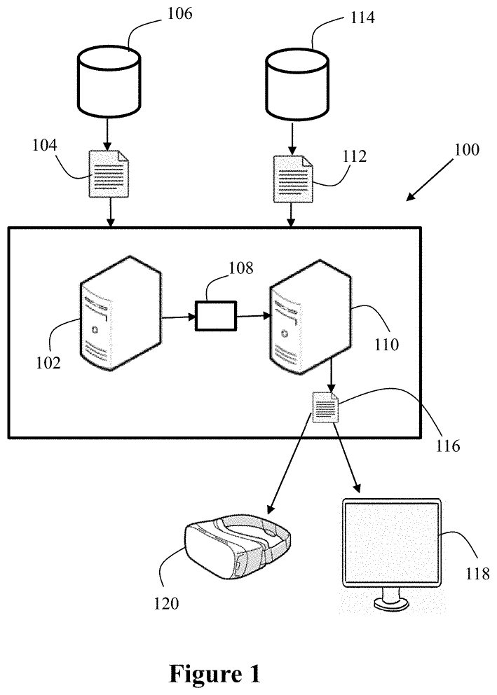

FIG. 1 illustrates an example AI Surgical Planning system 100 that leverages a prebuilt MD6DM model in order to enable machine learning and artificial intelligence algorithms to identify parameters in preparation for performing a surgical procedure. The AI Surgical Planning system 100 includes a training computer 102 that receives as input historical surgical data 104 of surgical procedures performed. The training computer 102 may receive the historical data 104 from a historical data store 106, for example. In one example, the training computer 102 may receive the historical data 104 from a multiple data sources (not shown). For example, the training computer 102 may be networked with multiple hospital systems, computers, or data stores and be set up to receive historical data 104 of surgical procedures performed by a variety of surgeons at a variety of hospitals in a variety of locations. Thus, by receiving as input historical data 104 from a variety of sources and therefore having access to a more diverse data set, the training computer 102 may function in a more robust manner and enable the AI Surgical Planning system 100 to identify parameters more accurately as compared to when the training computer 102 has access to a less diverse data set. Historical data 104 may include, for example, information about a surgical procedure specific to a patient, the parameters used/selected for the specific surgical procedure, and the outcome of the surgical procedure for that patient.

The training computer 102 also trains or learns based on the received historical data 104 and generates a recommendation algorithm 108 for identifying and recommending parameters for performing a surgical procedure. In particular, the training computer 102 analyzes the historical data 104 to understand the scenario surrounding many surgical procedures, the parameters chosen for the individual procedures, as well as the outcomes of the surgical procedure. Based on the analyses and what the training computer 102 has learned from the historical data 104, the training computer 102 generates the recommendation algorithm 108 which is able to process a data with respect to a new surgical procedure and to identify or suggest parameters for performing that new surgical procedure.

The AI Surgical Planning system 100 further includes a processing computer 110 that uses the recommendation algorithm 108 to identify and recommend parameters for a new surgical procedure. In particular, the processing computer 110 is configured to receive current surgical procedure data 112, or data with respect to a surgical procedure that is to be performed and for which the identification and recommendation of surgical procedure parameters is desired. The current surgical procedure data 112 may be received from a suitable source such as a current data store 114. The processing computer 110 processes the current surgical procedure data 112 using the recommendation algorithm 108 and determines parameters 116 for the new surgical procedure. The processing computer 110 is further configured to output the parameters or recommendations 116 to display 118, an HMD 120, or via another suitable peripheral (not shown). In one example, the processing computer 110 is configured to store the parameters 116 in the historical data store 106 so that the training computer 102 may continue to further train and refine the recommendation algorithm 108 based on additionally acquired or developed surgical procedure data.

It should be appreciated that surgical data, such as historical surgical data 104 and current surgical data 112, may include any suitable data that describes or provides information about a surgical procedure specific to a patient's anatomy. In one example, the surgical data can include a MD6DM model representative of a specific patient's anatomy. It should be further appreciated that although the training computer 102 and the processing computer 110 are illustrated as two distinct computing systems, the features and the functionality of the training computer 102 and the processing computer 110 may also be combined into a single computing system.

The training computer 102 and the processing computer 110 may be configured to leverage one or more AI machine learning algorithms to perform the functions as described. A machine learning algorithm may include a supervised learning algorithm in which both data input as well as a desired output is provided. One example of a supervised machine learning algorithm is Support Vector Machine in which the algorithm learns different classes based on historical data so that new data can be classified appropriately. Naïve Bayes Classifier is an example of a supervised machine learning algorithm that classifies data, specifically by applying the Bayes theorem. Another example of a supervised machine learning algorithm is Decision Tree in which a branching method is used to go from observations to conclusions in a predictive model approach. In one example, a machine learning algorithm is implemented as an Artificial Neural Network.

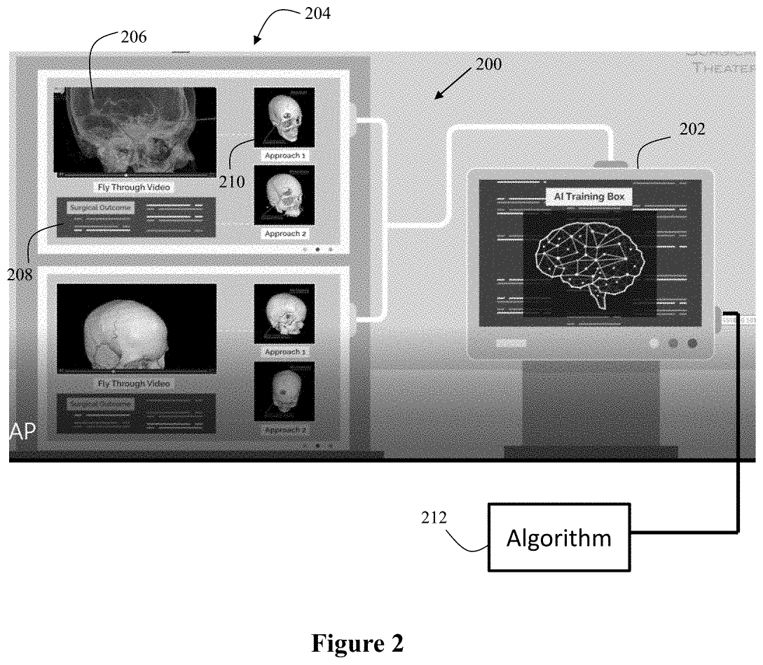

In one example, the AI Surgical Planning system 100 may be configured specifically for identifying and recommending parameters for performing a craniotomy, such as identifying an entry point and a trajectory for performing a craniotomy. FIG. 2 illustrates AI Surgical Planning system for craniotomy 200. The AI Surgical Planning system 200 includes a training computer 202, (e.g. the training computer 102 of FIG. 1) for receiving historical craniotomy data 204 and learning from the historical craniotomy data 204 in order to generate a craniotomy parameters algorithm 212 for identifying parameters for performing a craniotomy. In particular, the craniotomy data 204 may include, for example, a MD6DM model of a patient and illustrating a region of the patient's brain where a surgical procedure was performed. In one example, the craniotomy data 204 may include data representative of the surgical outcome 208. In one example, the craniotomy data 204 may include data representative of one or more approaches 210 or parameters that may have been contemplated for the craniotomy, including the selected approach (e.g. the entry point and trajectory).

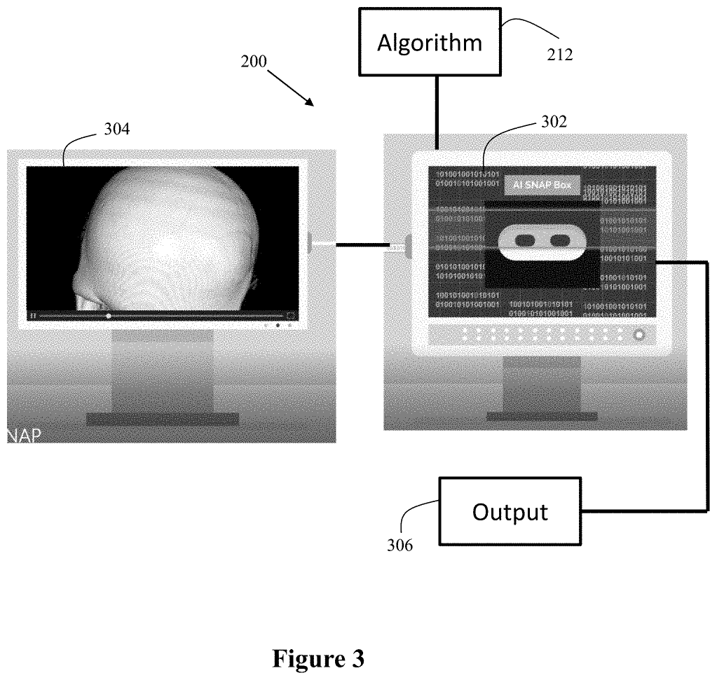

As illustrated in FIG. 3, AI Surgical Planning system for craniotomy 200 further includes a processing computer 302 (e.g. the processing computer 110 of FIG. 1) for leveraging the craniotomy parameters algorithm 212 generated by the training computer 200 to generate craniotomy parameters output 306. In particular, the processing computer 302 receives information about a patient and an MD6DM model of a patient that illustrates a region of the patient's brain where a surgical procedure is to be performed. The processing computer 302 applies the craniotomy parameters algorithm 212 in order to select an optimal approach for performing a craniotomy on the skull of the patient represented by the MD6DM model of the input data 304. In one example, the output 306 includes a visualization of the selected entry point and trajectory, via a HMD, either in a virtual view overlaid within the MD6DM or in an augmented reality view overlaid on top of an actual view of the patient.

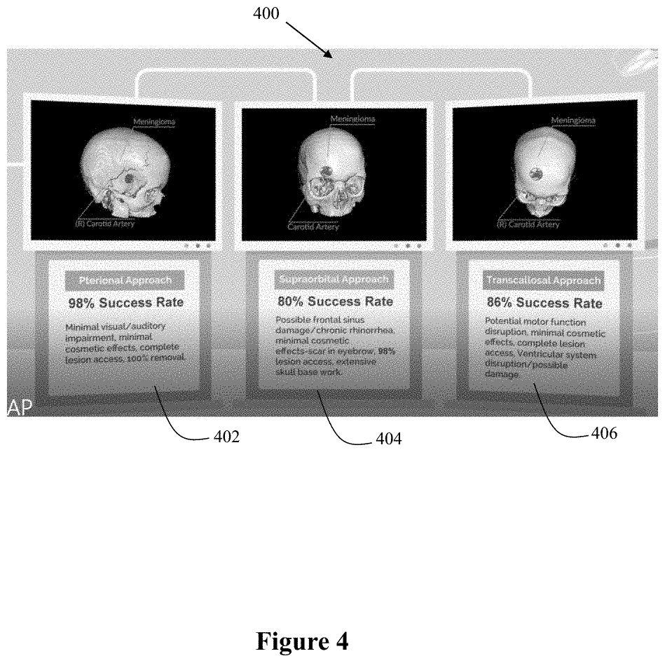

In one example, the output 306 includes, as illustrated in FIG. 4, a recommendation user interface 400 for providing multiple recommendations or suggestions for parameters, as apposed to selecting a single parameter or set of parameters. For example, the processing computer 302 may provide, via the recommendation user interface 400, several different approaches along with a calculated success rate, based on the AI Surgical Planning system's 200 knowledge learned from historical craniotomy data 204. In particular, the processing computer 302 may recommend, via the recommendation user interface 400, that a Pterional approach may have a 98% success rate in a first recommendation window 402, that a Supraorbital approach may have am 80% success rate in a second recommendation window 404, and that a Transcallosal approach may have an 86% success rate in a third recommendation window 406. The recommendation windows 402, 404, and 406, may each include respective descriptions, diagrams, and other suitable information for aiding in selecting the proper approach or parameters for performing the craniotomy.

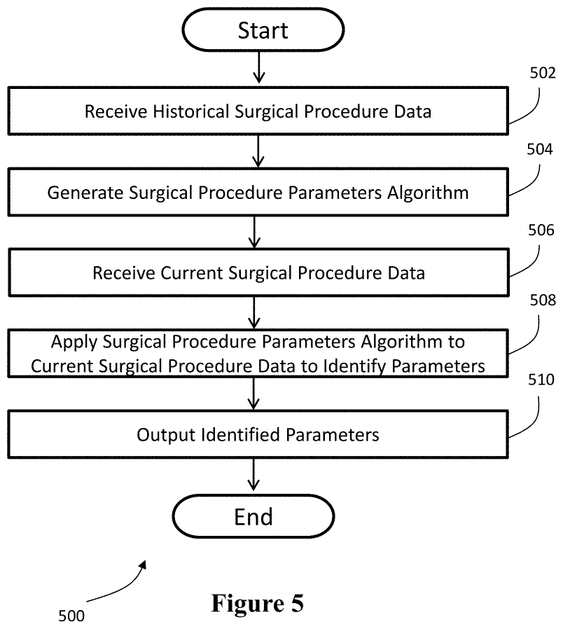

FIG. 5 illustrates an example method for determining parameters for a surgical procedure. At 502, an AI Surgical Planning system (e.g. the AI Surgical Planning system of FIG. 1) receives as input historical surgical procedure data. At 504, the AI Surgical Planning system generates a surgical procedures parameters algorithm using one or more artificial intelligence machine learning algorithms based on the received historical surgical procedure data. At 506, the AI Surgical Planning system receives current surgical procedure data for a specific patient for which a surgical procedure is to be performed. At 508, the AI Surgical Planning system applies the generated surgical procedures parameters algorithm to the received current surgical procedure data in order to determine surgical parameters for the surgical procedure to be performed for the specific patient. At 510, the AI Surgical Planning system outputs the identified parameters.

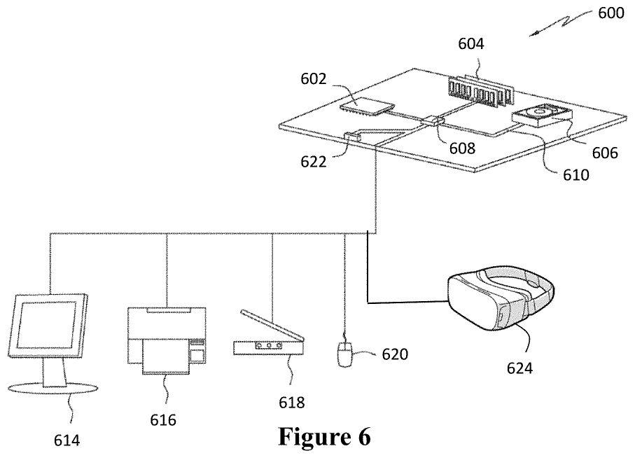

FIG. 6 is a schematic diagram of an example computer for implementing the training computer 102 and the processing computer 110 of FIG. 1. The example computer 600 is intended to represent various forms of digital computers, including laptops, desktops, handheld computers, tablet computers, smartphones, servers, and other similar types of computing devices. Computer 600 includes a processor 602, memory 604, a storage device 606, and a communication port 608, operably connected by an interface 610 via a bus 612.

Processor 602 processes instructions, via memory 604, for execution within computer 600. In an example embodiment, multiple processors along with multiple memories may be used.

Memory 604 may be volatile memory or non-volatile memory. Memory 604 may be a computer-readable medium, such as a magnetic disk or optical disk. Storage device 606 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 604 or storage device 606.

Computer 600 can be coupled to one or more input and output devices such as a display 614, a printer 616, a scanner 618, a mouse 620, and a HMD 624.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. An artificial intelligence surgical planning system comprising:
a display; and
a computer comprising one or more processors, one or more computer-readable tangible storage devices, and program instructions stored on at least one of the one or more storage devices for execution by at least one of the one or more processors, the program instructions being configured to:
receive as input historical surgical procedure data relating to a plurality of surgical procedures previously performed for a plurality of patients;
generate a surgical procedures parameters algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data;
receive current surgical procedure data for a patient for which a surgical procedure is to be performed;
apply the generated surgical procedures parameters algorithm to the received current surgical procedure data in order to identify a recommended surgical parameter for the surgical procedure to be performed for the current patient; and
output the recommended surgical parameter to the display.

2. The artificial intelligence surgical planning system of claim 1, wherein the computer is networked with a plurality of data sources and configured to receive historical data of surgical procedures performed by a plurality of surgeons at a plurality of hospitals in a plurality of locations.

3. The artificial intelligence surgical planning system of claim 1, wherein the historical surgical procedure data comprises at least one of information about a surgical procedure specific to a patient, a parameter used for a specific surgical procedure, and an outcome of the surgical procedure for a patient.

4. The artificial intelligence surgical planning system of claim 1, wherein the surgical procedures parameters algorithm is configured for identifying a recommended parameter for performing a craniotomy.

5. The artificial intelligence surgical planning system of claim 5, wherein the recommended parameter comprises an entry point and a trajectory.

6. The artificial intelligence surgical planning system of claim 1, wherein the display comprises an augmented reality head mounted display, and wherein the computer is configured to output the recommended surgical parameter by overlaying the recommended surgical parameter on top of an actual view of the current patient.

7. The artificial intelligence surgical planning system of claim 1, wherein the surgical procedures parameters algorithm is configured to identify a plurality of recommended surgical parameters for a current patient and to calculate a corresponding success rate of each of the plurality of recommended surgical parameters based on the historical surgical procedure data, and wherein the computer is configured to output the plurality of recommended surgical parameters and the corresponding success rates to the display.

8. A method for identifying a recommended surgical parameter for a surgical procedure, comprising the steps of:
receiving as input historical surgical procedure data relating to a plurality of surgical procedures previously performed for a plurality of patients;
generating a surgical procedures parameters algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data;
receiving current surgical procedure data for a patient for which a surgical procedure is to be performed;
applying the generated surgical procedures parameters algorithm to the received current surgical procedure data in order to identify a recommended surgical parameter for the surgical procedure to be performed for the current patient; and
outputting the recommended surgical parameter to a display.

9. The method of claim 8, wherein receiving as input historical surgical procedure data comprises receiving historical data of surgical procedures performed by a plurality of surgeons at a plurality of hospitals in a plurality of locations.

10. The method of claim 8, wherein the historical surgical procedure data comprises at least one of information about a surgical procedure specific to a patient, a parameter used for a specific surgical procedure, and an outcome of the surgical procedure for a patient.

11. The method of claim 8, wherein the surgical procedures parameters algorithm is configured for identifying a recommended parameter for performing a craniotomy.

12. The method of claim 11, wherein the recommended parameter comprises an entry point and a trajectory.

13. The method of claim 8, wherein outputting the recommended surgical parameter to a display comprises outputting the recommended surgical parameter to an augmented reality head mounted display and overlaying the recommended surgical parameter on top of an actual view of the current patient.

14. The method of claim 8, wherein the surgical procedures parameters algorithm is configured to identify a plurality of recommended surgical parameters for a current patient and to calculate a corresponding success rate of each of the plurality of recommended surgical parameters based on the historical surgical procedure data, and wherein outputting the recommended surgical parameter to a display comprises outputting the plurality of recommended surgical parameters and the corresponding success rates to the display.

15. A method for identifying a recommended surgical parameter for a surgical procedure, comprising the steps of:
receiving as input historical surgical procedure data relating to a plurality of surgical procedures previously performed for a plurality of patients, said surgical procedure data including information about a craniotomy procedure specific to a patient;
generating a surgical procedures parameters algorithm that is configured for identifying a recommended parameter including both an entry point and a trajectory for performing a craniotomy, said algorithm using one or more artificial intelligence machine learning algorithms based on the received historical surgical procedure data, wherein the surgical procedures parameters algorithm is configured to identify recommended a surgical parameter for a surgical procedure to be performed for a current patient based on current surgical procedure data;
receiving current surgical procedure data for the patient for which a surgical procedure is to be performed;
applying the generated surgical procedures parameters algorithm to the received current surgical procedure data in order to identify a recommended surgical parameter for the surgical procedure to be performed for the current patient; and
outputting the recommended surgical parameter to an augmented reality head mounted display and overlaying the recommended surgical parameter on top of an actual view of the current patient.

16. The method of claim 15, wherein the surgical procedures parameters algorithm is also configured to identify a plurality of recommended surgical parameters for a current patient including both an entry point and a trajectory, said surgical procedures parameters algorithm also configured to calculate a corresponding success rate of each of the plurality of recommended surgical parameters based on the historical surgical procedure data, and wherein outputting the recommended surgical parameter to a display comprises outputting the plurality of recommended surgical parameters and the corresponding success rates to the display.

17. The method of claim 16, wherein receiving as input historical surgical procedure data comprises receiving historical data of surgical procedures performed by a plurality of surgeons at a plurality of hospitals in a plurality of locations.

18. The method of claim 15, wherein receiving as input historical surgical procedure data comprises receiving historical data of surgical procedures performed by a plurality of surgeons at a plurality of hospitals in a plurality of locations.
